(19) United States
(12) Patent Application Publication
(10) Pub. No.: US 2024/0366580 A1
(43) Pub. Date: Nov. 7, 2024

(54) NOVEL PHARMACEUTICAL COMPOSITIONS COMPRISING DABIGATRAN ETEXILATE OR SALT THEREOF

(71) Applicant: Zim Laboratories Limited, Kalmeshwar, Nagpur (IN)

(72) Inventors: Chandrashekhar Mainde, Kalmeshwar, Nagpur (IN); Uttam Kedar, Kalmeshwar, Nagpur (IN); Kuldeep Gangawat, Kalmeshwar, Nagpur (IN)

(73) Assignee: Zim Laboratories Limited, Kalmeshwar, Nagpur (IN)

(21) Appl. No.: 18/566,931

(22) PCT Filed: Jun. 1, 2022
(86) PCT No.: PCT/IB2022/055118

§ 371 Date: Dec. 4, 2023

(30) Foreign Application Priority Data
Jun. 4, 2021 (IN) 202121024864

(51) Int. Cl. A61K 31/4439; A61K 9/48; A61K 31/661

(52) U.S. Cl. CPC A61K 31/4439; A61K 9/485; A61K 9/4858; A61K 9/4866; A61K 31/661

(57) ABSTRACT

The present invention relates to novel pharmaceutical compositions comprising dabigatran etexilate or salt thereof for the prophylaxis and treatment of Deep Venous Thrombosis and Pulmonary Embolism. The pharmaceutical composition comprising dabigatran etexilate or salt thereof, glycerophosphoric acid (GPA) along with one or more pharmaceutically acceptable excipient.

### FIELD OF THE INVENTION

The present invention relates to novel pharmaceutical compositions comprising dabigatran etexilate or salt thereof in the prophylaxis and treatment of Deep Venous Thrombosis and Pulmonary Embolism. Specifically present invention relates to pharmaceutical composition comprising dabigatran etexilate or salt thereof and glycerophosphoric acid (GPA) and process of manufacturing the same.

### BACKGROUND OF THE INVENTION

Anticoagulants, commonly known as blood thinners, are chemical substances that prevent or reduce coagulation of blood and prolonging the clotting time. Anticoagulants are used in therapy for thrombotic disorders. Traditionally warfarin, coumarins, and heparins are used as Anticoagulants. Recently a number of agents has been introduced, these include direct thrombin inhibitors like dabigatran and factor Xa inhibitors like rivaroxaban, apixaban, betrixaban and edoxaban.

Dabigatran etexilate mesylate is a direct thrombin inhibitor. Chemically it is β-Alanine, N-[[2-[[[4-[[[(hexyloxy) carbonyl] amino]iminomethyl] phenyl]amino]methyl]-1-methyl-1H-benzimidazol-5-yl]carbonyl]-N-2-pyridinyl-,ethyl ester, methanesulfonate with a molecular weight of 723.86, a molecular formula of C₃₄H₄₁N₇O₅·CH₄O₃S and Dabigatran etexilate mesylate is represented by compound of structural formula I.

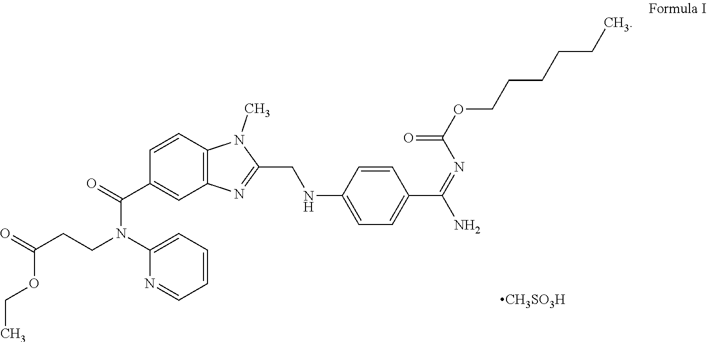

Dabigatran etexilate mesylate is a yellow-white to yellow powder. A saturated solution in pure water has a solubility of 1.8 mg/ml. It is freely soluble in methanol, slightly soluble in ethanol and sparingly soluble in isopropanol.

Dabigatran etexilate mesylate Capsule was approved in USA on Oct. 19, 2010 under the tradename PRADAXA® and is available in the strength of Eq 75 mg base, Eq 110 mg base and 150 mg base. The product is indicated for the Reduction of Risk of Stroke and Systemic Embolism in Non-valvular Atrial Fibrillation, Treatment of Deep Venous Thrombosis and Pulmonary Embolism, Reduction in the Risk of Recurrence of Deep Venous Thrombosis and Pulmonary Embolism and Prophylaxis of Deep Vein Thrombosis and Pulmonary Embolism Following Hip Replacement Surgery.

Dabigatran etexilate mesylate Capsules under the trade name PRADAXA® contain dabigatran etexilate mesylate as the active ingredient and inactive ingredients as acacia, dimethicone, hypromellose, hydroxypropyl cellulose, talc and tartaric acid. The capsule shell is composed of carrageenan, hypromellose, potassium chloride, titanium dioxide, black edible ink and FD&C Blue No.

U.S. Pat. No. 6,087,380 discloses compound Dabigatran Etexilate or salt thereof.

U.S. Pat. No. 7,932,273 discloses polymorph form II of Dabigatran etexilate mesylate.

U.S. Pat. No. 9,925,174 discloses pharmaceutical composition of Dabigatran etexilate mesylate along with tartaric acid or fumaric acid or succinic acid or citric acid or malic acid or glutamic acid or aspartic acid and any combination thereof.

U.S. Ser. No. 10/251,840 discloses method of preparing insulated tartaric acid pellets 3, comprising a first step of preparing tartaric acid pellets 1 comprising alternatively spraying tartaric acid particles with a solution of tartaric acid and a binder followed by sprinkling the tartaric acid particles with a fine tartaric acid powder having a particle size of <100 microns, wherein at least 90% of the tartaric acid particles have a particle size in the range from 0.4-0.6 mm, and in a second step, spraying an ethanolic insulating suspension 2 comprising hydroxypropylmethylcellulose onto the tartaric acid pellets 1 to form insulated tartaric acid pellets 3 having substantially uniform, sphere-like shapes.

Dabigatran Etexilate Mesylate (DEM) is a salt form of the Dabigatran Etexilate Prodrug. It is absorbed and transformed by esterase catalyzed hydrolysis in the liver after oral administration to dabigatran. It is a BCS Class II drug having high permeability, low solubility. The low solubility of drug is responsible for low bioavailability. It is having absolute bioavailability of 3-7% followed by oral administration. It is prone to the acidic condition and is degraded in the presence of moisture by hydrolytic processes. Dabigatran Etexilate Mesylate aqueous solubility depends on pH with elevated solubility in acidic media and very low solubility in neutral and basic media.

The commercial available product and product known in the prior art for Dabigatran Etexilate Mesylate contains tartaric acid or fumaric acid or succinic acid or citric acid or malic acid or glutamic acid or aspartic acid and any combination thereof. Preferably the product in the prior art contains tartaric acid. The Dabigatran Etexilate Mesylate or oral anticoagulant are associated with gastric adverse reactions like abdominal pain, abdominal discomfort and epigastric discomfort, GERD, esophagitis, erosive gastritis, gastric hemorrhage, hemorrhagic gastritis, hemorrhagic erosive gastritis and gastrointestinal ulcer. Further product containing Dabigatran Etexilate Mesylate and strong acid like tartaric acid worsen the gastric adverse reactions by promoting GI bleeding through caustic injury; therefore leading to discontinuation of Dabigatran therapy.

Also the commercial available product and product known in the prior art teaches to overcome gastrointestinal hemorrhage and other gastric adverse reaction by concomitant use of proton pump inhibitors like pantoprazole. However, use of proton pump inhibitors like pantoprazole increases the alkalinity in the GI tract; therefore decrease in solubility, absorption of Dabigatran Etexilate Mesylate and results in lesser bioavailability.

Accordingly, by considering physiochemical and biopharmaceutical properties of Dabigatran Etexilate or salt thereof, applicant of present invention invented novel pharmaceutical compositions comprising dabigatran etexilate or salt thereof and glycerophosphoric acid (GPA); also the process of manufacturing the same.

Therefore, novel pharmaceutical compositions comprising dabigatran etexilate or salt thereof and glycerophosphoric acid (GPA) according to present invention provides more stability, bioavailability with less gastric adverse reactions in reduction of risk of stroke and systemic embolism in Non-valvular Atrial Fibrillation, Treatment of Deep Venous Thrombosis and Pulmonary Embolism, Reduction in the Risk of Recurrence of Deep Venous Thrombosis and Pulmonary Embolism and Prophylaxis of Deep Vein Thrombosis and Pulmonary Embolism Following Hip Replacement Surgery. Also the novel pharmaceutical composition can be administered without concomitant use of proton pump inhibitors.

### OBJECT OF THE INVENTION

Accordingly, it is an object of this invention is to provide novel pharmaceutical compositions comprising dabigatran etexilate or salt thereof and glycerophosphoric acid (GPA) in the prophylaxis and treatment of Deep Venous Thrombosis and Pulmonary Embolism.

Another object of this invention is to provide novel pharmaceutical compositions comprising dabigatran etexilate or salt thereof and glycerophosphoric acid (GPA) which provides less gastric adverse reactions in the prophylaxis and treatment of Deep Venous Thrombosis and Pulmonary Embolism.

A further object of this invention is to provide novel pharmaceutical compositions comprising dabigatran etexilate or salt thereof and glycerophosphoric acid (GPA) which provides less gastric adverse reactions and can be administered without concomitant use of proton pump inhibitors in the prophylaxis and treatment of Deep Venous Thrombosis and Pulmonary Embolism.

A further object of this invention is to provide novel pharmaceutical compositions comprising dabigatran etexilate or salt thereof and glycerophosphoric acid (GPA) which provides more bioavailability and patient compliance in the prophylaxis and treatment of Deep Venous Thrombosis and Pulmonary Embolism.

A further object of this invention is to provide novel pharmaceutical compositions comprising dabigatran etexilate or salt thereof, glycerophosphoric acid (GPA) along with one or more pharmaceutically acceptable excipient wherein glycerophosphoric acid containing core and dabigatran layer is separated by seal coating and sugar coating.

A further object of this invention is to provide process of manufacturing novel pharmaceutical compositions comprising dabigatran etexilate or salt thereof, glycerophosphoric acid (GPA) along with one or more pharmaceutically acceptable excipient; wherein the step comprises preparing the central core of glycerophosphoric acid (GPA) along with one or more excipient followed by seal coating, sugar coating, dabigatran layer coating and seal coating.

### SUMMARY OF THE INVENTION

A first aspect of the present invention is to provide novel pharmaceutical composition comprising dabigatran etexilate or salt thereof and glycerophosphoric acid (GPA).

In another aspect of the present invention is to provide novel pharmaceutical composition comprising dabigatran etexilate or salt thereof, glycerophosphoric acid (GPA) along with one or more pharmaceutically acceptable excipient.

In another aspect of the present invention is to provide novel pharmaceutical composition comprising dabigatran etexilate or salt thereof, glycerophosphoric acid (GPA) along with one or more pharmaceutically acceptable excipient; wherein composition having central core of inactive ingredient followed by seal coating, sugar coating, drug layering and seal coating.

In another aspect of the present invention is to provide process of manufacturing novel pharmaceutical compositions comprising dabigatran etexilate or salt thereof, glycerophosphoric acid (GPA) along with one or more pharmaceutically acceptable excipient wherein the steps comprises
i. preparing central core by using glycerophosphoric acid (GPA) along with one or more pharmaceutically acceptable excipient.
ii. providing seal coating on the central core by coating excipient.
iii. providing sugar coating on the seal coated core in step ii.
iv. applying dabigatran etexilate or salt thereof layering on the sugar coated core in step iii.
v. Providing seal coating on the dabigatran etexilate or salt thereof layer obtained in step iv.

In another aspect of the present invention is to provide novel pharmaceutical compositions comprising dabigatran etexilate or salt thereof, glycerophosphoric acid (GPA) along with one or more pharmaceutically acceptable excipient in the prophylaxis and treatment of Deep Venous Thrombosis and Pulmonary Embolism.

### DETAIL DESCRIPTION OF THE INVENTION

The present invention relates to novel pharmaceutical compositions comprising dabigatran etexilate or salt thereof for the prophylaxis and treatment of Deep Venous Thrombosis and Pulmonary Embolism.

In another aspect of the present invention is to provide novel pharmaceutical compositions comprising dabigatran etexilate or salt thereof and glycerophosphoric acid (GPA).

The novel pharmaceutical compositions according to present invention comprises dabigatran etexilate or salt thereof, glycerophosphoric acid (GPA) along with one or more pharmaceutically acceptable excipient.

The dabigatran etexilate according to present invention may be present in its base form or its pharmaceutically acceptable salt form. Preferably dabigatran etexilate is present in its mesylate form i.e. dabigatran etexilate mesylate.

The novel pharmaceutical compositions according to present invention contain dabigatran etexilate or salt thereof in the range from 10 mg to 300 mg; preferably 50 mg to 200 mg. More preferably 75 mg, 110 mg and 150 mg equivalent to dabigatran etexilate.

Glycerophosphoric acid (GPA) is organic acid and is colorless liquid. It is having molecular formula of C₃H₉O₆P and molecular weight of 172.07. The amount of Glycerophosphoric acid in the composition according to present invention ranges from 5 to 40%; more preferably 10 to 30% by weight of the compositions.

The dabigatran etexilate or salt thereof and Glycerophosphoric acid (GPA) according to present invention may be present in the ratio from 1:0.2 to 1:2 respectively.

The one or more pharmaceutically acceptable excipient according to present invention may be selected from the group consisting of diluent, disintegrant, binder, antioxidant, channeling agent, anti-adherent, antifoaming agent, seal coating polymer, solvent or combinations thereof.

The diluent includes but not limited to Microcrystalline Cellulose, Mannitol, Lactose Anhydrous, Lactose Monohydrate, Ammonium Alginate, Calcium Carbonate, Calcium Lactate, Anhydrous Dibasic Calcium Phosphate, Dibasic Calcium Phosphate Dihydrate, Tribasic Calcium Phosphate, Calcium Sulfate, Corn Starch, Pregelatinized Starch, Dextrates, Dextrin, Dextrose, Erythritol, Fructose, Glyceryl Palmitostearate, Kaolin, Lactitol, Maltitol, Magnesium Carbonate, Magnesium Oxide, Maltodextrin, Maltose, Polydextrose, Polymethacrylates, Simethicone, Sodium Chloride, Sorbitol, Starch, Pregelatinized Starch, Sucrose, Sulfobutylether b-Cyclodextrin, Talc, Trehalose, Xylitol or combination thereof and alike. Preferably diluent is Microcrystalline Cellulose. The composition according to present invention contains diluent from 10 to 75% by weight of composition.

The disintegrant includes but not limited to crosspovidone, sodium starch glycolate, croscarmellose sodium, Kollidon, magnesium aluminum silicate, Chitin, Mannitol, Crosslinked alginic acid, Crosslinked starch, Calcium silicate or combination thereof and alike. Preferably disintegrant is Crospovidone. The compositions according to present invention contains disintegrant from 1 to 10% by weight of composition.

The binder includes but not limited to Acacia, Hydroxypropyl cellulose Povidone, Agar, Calcium Carbonate, Tribasic Calcium Phosphate, Carbomer, Carboxymethylcellulose Calcium, Microcrystalline Cellulose, Powdered Cellulose, Ceratonia, Chitosan, Dextrin, Ethylcellulose, Gelatin, Liquid Glucose, Guar Gum, Hydroxyethyl Cellulose, Hydroxyethylmethyl Cellulose, Hypromellose, Inulin, Lactose Monohydrate, Magnesium Aluminum Silicate, Maltodextrin, Methylcellulose, Polycarbophil, Polydextrose, Polyethylene Oxide, Polymethacrylates, Sodium Alginate, Pregelatinized Starch, Starch, Sucrose, Sunflower Oil, Hydrogenated Vegetable Oil, Vitamin E Polyethylene Glycol Succinate, Zein or combination thereof and alike. Preferably binder is Acacia, Hydroxypropyl cellulose or combination thereof. The composition according to present invention contains binder from 2 to 20% by weight of composition.

The antioxidant includes but not limited to butylated Hydroxytoluene, butylated Hydroxyanisole, ascorbic acid, tocopherol, sodium ascorbate, propyl gallate or combination thereof and alike. Preferably antioxidant is butylated Hydroxytoluene, butylated Hydroxyanisole or combination thereof. The composition according to present invention contains antioxidant from 0.02 to 0.5% by weight of composition.

The channeling agent includes but not limited to Sucrose, sugar, sodium chloride, polyols or combination thereof and alike. Preferably channeling agent is Sucrose. The composition according to present invention contains channeling agent from 2 to 20% by weight of composition.

The anti-adherent includes but not limited to talc, cornstarch, metal stearates, sodium lauryl sulfate or combination thereof and alike. Preferably anti-adherent is talc. The composition according to present invention contains anti-adherent from 0.5 to 2% by weight of composition.

The antifoaming agent includes but not limited to Simethicone, cetostearyl alcohol, castor oil, stearates, polydimethylsiloxanes and other silicones derivatives, ether, glycols or combination thereof and alike. Preferably antifoaming agent is Simethicone. The composition according to present invention contains antifoaming agent from 0.2 to 1% by weight of composition.

The seal coating polymer includes but not limited to hydroxyl propyl methyl cellulose, hydroxyl propyl cellulose, methylcellulose, ethyl cellulose or combination thereof and alike. The most preferably seal coating polymer is hydroxyl propyl methyl cellulose. The composition according to present invention contains seal coating polymer from 2 to 30% by weight of composition.

The solvent includes but not limited to isopropyl alcohol, dichloromethane, water, ethanol, acetone, methanol, acetone, Tetrachloroethylene, Toluene, Methyl acetate, Ethyl acetate or combination thereof and alike. Preferably solvent is isopropyl alcohol, dichloromethane and water or combination thereof.

The novel pharmaceutical compositions according to present invention may be in the form of pellet, granule, tablet, capsule or bead.

In another aspect of the present invention is to provide novel pharmaceutical compositions comprising dabigatran etexilate or salt thereof, glycerophosphoric acid (GPA) along with one or more pharmaceutically acceptable excipient; wherein composition having central core of inactive ingredient followed by seal coating, sugar coating, drug layering and seal coating.

The central core according to present invention may be in the form of pellet, granule, bead, spheroid or tablets. The central core comprises glycerophosphoric acid (GPA) along with one or more pharmaceutically acceptable excipient. The central core may comprises glycerophosphoric acid (GPA), binder along with one or more pharmaceutically acceptable excipient.

The central core according to present invention is further coated with seal coating polymer to form the seal coating. The seal coating is further coated with sugar coating polymer to form the sugar coated coat. On the sugar coated layer, drug loading i.e. dabigatran etexilate or salt thereof loading is performed to form the dabigatran etexilate or salt layer. The drug layer is further coated with seal coating. The seal coatings according to present invention may be optional.

The drug layer of dabigatran etexilate or salt thereof according to present invention contains dabigatran etexilate or salt thereof along with one or more pharmaceutically acceptable excipient. The drug layer of dabigatran etexilate or salt thereof may comprises antioxidant, binder along with one or more pharmaceutically acceptable excipient.

The novel pharmaceutical composition comprising dabigatran etexilate or salt thereof according to present invention; wherein dabigatran etexilate or salt thereof layer is separated with central core containing glycerophosphoric acid (GPA) by seal coating followed by sugar coating. The said seal coating and sugar coating prevents contact of acid i.e. glycerophosphoric acid (GPA) with dabigatran etexilate or salt thereof which is prone to the acidic condition. In addition, the coating prevents hydrolytic degradation of dabigatran due to moisture.

In another aspect of the present invention is to provide process of manufacturing novel pharmaceutical composition comprising dabigatran etexilate or salt thereof, glycerophosphoric acid (GPA) along with one or more pharmaceutically acceptable excipient wherein the steps comprises
i. preparing central core by using glycerophosphoric acid (GPA) along with one or more pharmaceutically acceptable excipient.
ii. providing seal coating on the central core by coating excipient.
iii. providing sugar coating on the seal coated core in step ii.
iv. applying dabigatran etexilate or salt thereof layering on the sugar coated core in step iii.
v. Providing seal coating on the dabigatran etexilate or salt thereof layer obtained in step iv.

The central core according to present invention may be manufactured by using dry granulation or wet granulation or slugging method. The central core may be in the form of pellet, bead or spheroid, which is formed by extrusion spheronization method. The central core may be in the form of granules, the said granules may be optionally compressed to form the tablet.

In another embodiment of the present invention is to provide process of manufacturing novel pharmaceutical compositions comprising dabigatran etexilate or salt thereof, glycerophosphoric acid (GPA) along with one or more pharmaceutically acceptable excipient wherein the steps comprises
i. preparing central core by using glycerophosphoric acid (GPA) along with one or more pharmaceutically acceptable excipient.
ii. optionally providing seal coating on the central core by coating excipient.
iii. providing sugar coating on the seal coated core in step ii.
iv. applying dabigatran etexilate or salt thereof layering on the sugar coated core in step iii.
v. Providing seal coating on the dabigatran etexilate or salt thereof layer obtained in step iv.

In yet another embodiment of the present invention is to provide process of manufacturing novel pharmaceutical compositions comprising dabigatran etexilate or salt thereof, glycerophosphoric acid (GPA) along with one or more pharmaceutically acceptable excipient wherein the steps comprises
i. preparing central core by using glycerophosphoric acid (GPA) along with one or more pharmaceutically acceptable excipient.
ii. providing sugar coating on the central core.
iii. applying dabigatran etexilate or salt thereof layering on the sugar coated core in step ii.
iv. Providing seal coating on the dabigatran etexilate or salt thereof layer obtained in step iii.

In yet another embodiment of the present invention is to provide process of manufacturing novel pharmaceutical compositions comprising dabigatran etexilate or salt thereof, glycerophosphoric acid (GPA) along with one or more pharmaceutically acceptable excipient wherein the steps comprises
i. preparing central core by using glycerophosphoric acid (GPA) along with one or more pharmaceutically acceptable excipient.
ii. providing seal coating on the central core by coating excipient.
iii. applying dabigatran etexilate or salt thereof layering on the seal coated core in step ii.
iv. Providing seal coating on the dabigatran etexilate or salt thereof layer obtained in step iii.

In yet another embodiment is to provide process of manufacturing novel pharmaceutical compositions comprising dabigatran etexilate or salt thereof, glycerophosphoric acid (GPA) along with one or more pharmaceutically acceptable excipient wherein the steps comprises
i. preparing central core by using glycerophosphoric acid (GPA) along with Microcrystalline Cellulose, Crospovidone, Acacia and optionally purified water.
ii. Providing seal coating on the central core by coating solution comprising HPMC, Isopropyl Alcohol and Dichloromethane.
iii. providing sugar coating on the seal coated core in step ii by sugar coating solution comprising Sucrose, Talc, Purified Water and Isopropyl Alcohol.
iv. applying dabigatran etexilate or salt thereof layer on the sugar coated core in step iii optionally along with one or more pharmaceutically acceptable excipient.
v. Providing seal coating on the dabigatran etexilate or salt thereof layer obtained in step iv by coating solution comprising HPMC, Isopropyl Alcohol, Simethicone and Dichloromethane.

The central core according to present invention prepared by using glycerophosphoric acid (GPA) along with Microcrystalline Cellulose, Crospovidone, Acacia and purified water, can be formed by wet granulation method followed by extrusion spheronization to form pellet, bead or spheroid.

The central core according to present invention prepared by using glycerophosphoric acid (GPA) along with Microcrystalline Cellulose, Crospovidone, Acacia and optionally purified water or solvent can involve dry or wet granulation method to form granule; which may optionally compressed into the tablet.

The novel pharmaceutical composition according to present invention in the form of pellet, granule, bead or spheroid may be filled into the capsule or sachet.

Dabigatran etexilate has low solubility in water, so dissolution is most likely the rate limiting step for absorption. Solubility is strongly pH dependent with increased solubility at acidic pH. Therefore glycerophosphoric acid (GPA) according to present invention provides acidic environment required for dissolution and absorption of Dabigatran etexilate. The amount of dabigatran etexilate or salt thereof, glycerophosphoric acid (GPA) along with one or more pharmaceutically acceptable excipient as well process of manufacturing i.e. formation of central core of glycerophosphoric acid (GPA), seal coating, sugar coating, drug layering in the composition has been optimized in such way that, composition according to present invention provides desired release of the dabigatran etexilate from the dosage form in the prophylaxis and treatment of Deep Venous Thrombosis and Pulmonary Embolism. Further the weight ratio of dabigatran etexilate or salt thereof, glycerophosphoric acid (GPA) and release profile of dabigatran etexilate from the dosage form according to present invention provides more bioavailability, less adverse effect and better patient compliance in the prophylaxis and treatment of Deep Venous Thrombosis and Pulmonary Embolism. Therefore the composition according to present invention can be administered without concomitant use of proton pump inhibitors.

The dissolution of the test product according to present invention and reference product Pradaxa 150 mg has been performed in 0.01 N HCl, USP Type I, (Basket with modified diameter 24.5 mm) apparatus at 100 rpm. The results of dissolution of test product has been found to be in compliance with that of reference product.

The novel pharmaceutical composition according to present invention is in the form of pellet, granule, bead, spheroid or tablet can be packaged in suitable airtight containers and moisture proof packs. Packaging may include but not limited to high-density polyethylene bottle, aluminum blister package. The container material or packaging material of the present invention does not affect the quality of the preparation or does not allow diffusion of any kind into or across the material of the container into the preparation.

### EXAMPLE

Some illustrative non-limiting example of the present invention are described below

| | | |
|---|---|---|
| Sr. No. | Ingredients | Mg/unit |

Stage I: Granulation, Extrusion and Spheronization

| | | |
|---|---|---|
| 1. | Microcrystalline Cellulose | 99.00 |
| 2. | Crospovidone | 8.00 |
| 3. | Glycerophosphoric Acid | 65.00 |
| 4. | Acacia | 8.00 |
| 5. | Purified Water | Qs |

Stage II: Seal Coating

| | | |
|---|---|---|
| 6. | HPMC 5 cps | 9.00 |
| 7. | Isopropyl Alcohol | Qs |
| 8. | Dichloromethane | Qs |

Stage III: Sugar coating

| | | |
|---|---|---|
| 9. | Sucrose | 23.63 |
| 10. | Talc | 4.73 |
| 11. | Purified Water | Qs |
| 12. | Isopropyl Alcohol | Qs |

Stage IV: Drug loading

| | | |
|---|---|---|
| 13. | Dabigatran EM | 172.95 |
| 14. | BHA | 0.25 |
| 15. | BHT | 0.25 |
| 16. | Simethicone | 8.65 |
| 17. | HPC 85 | 8.65 |
| 18. | Isopropyl Alcohol | Qs |
| 19. | Dichloromethane | Qs |

Stage V: Seal coating

| | | |
|---|---|---|
| 20. | HPMC 5 cps | 18.54 |
| 21. | Simethicone | 1.85 |
| 22. | Isopropyl Alcohol | Qs |
| 23. | Dichloromethane | Qs |

| | |
|---|---|
| Total | 428.50 |

Stage VI: Filling

| | |
|---|---|
| 24. | HPMC Capsule shell size “0” Blue/White |

Stage VI: Packaging

| | | |
|---|---|---|
| 1. | Alu Alu blister pack | Packaging material |
| 2. | HDPE BOTTLES 60 CC/33 MM | Packaging material |

### Manufacturing Process

### 1. Granulation, Extrusion and Spheronization

1.1 Co-Sift microcrystalline Cellulose, Crospovidone, and Acacia through #40 ASTM.
1.2 Preparation of granulating solution by addition of Glycerophosphoric acid in Water.
1.3 Granulate the step 1.1 content in rapid mixture granulator (RMG) by using step 1.2 solutions.
1.4 Pass wet granules of 1.3 through Extrusion (Screen 0.8 mm) followed by Spheronization.
1.5. Drying of pellets in Try dryer at 45-55° C. and sifting through (20 #-25 #)

### 2. Seal Coating

2.1 Disperse HPMC (5 CPS) in Isopropyl Alcohol under stirring.
2.2 Then add Dichloromethane in above solution and filter through nylon cloth.
2.3 Use the above solution for coating of pellets using Fluidized bed coater
2.4 Dry the coated pellet in Fluidized bed coater (FBC) at 40-45° C. and sift through (20 #-25 #).

### 3. Sugar Coating

3.1 Dissolve Sucrose in Water under stirring.
3.2 Dilute the solution by using Isopropyl alcohol then add talc and filter through nylon cloth.
3.3 Use the above solution for coating of seal coated pellet using Fluidized bed coater
3.4 Dry the sugar coated pellet in FBC at 45-55° C. and sift through (18 #-20 #).

### 4. Drug Loading

4.1 Dissolve Butylated Hydroxyanisole, Butylated Hydroxytulene, and Hydroxypropyl cellulose 85 in Isopropyl alcohol under stirring.
4.2 Dissolve Simethicone in Dichloromethane then add above solution under stirring
4.3 Add Dabigatran EM in above solution under stirring.
4.4 Filter the dispersion by using nylon cloth.
4.5 Use FBC for drug loading.
4.6 Air drying the drug-loaded pellet in FBC and sift through (14 #-20 #).

### 5. Seal Coating

5.1 Disperse HPMC (5 CPS) in Isopropyl Alcohol under stirring.
5.2 Dissolved Simethicone in Dichloromethane then add in above solution and filter through nylon cloth.
5.3 use FBC for the seal coating.
5.4 Dry the seal coated pellet in FBC at 40-45° C. and sift through (14 #-20 #).

### 6. Capsules Filling

6.1 HPMC Capsule shell Size “0” Blue cap and white body filled with yellow to light yellow free flow pellets.

### Dissolution Profile for Example

Test Product (Tₜ): Dabigatran Etexilate Capsules 150 mg
Reference Product (Rₜ): Pradaxa 150 mg
Dissolution Medium: 0.01 N HCl, USP Type I, (Basket with modified diameter 24.5 mm), 100 rpm.

TABLE 2
Dissolution profile for Example:

| | Time | Average % drug release | | | |
|---|---|---|---|---|---|
| Sr. | Interval | Rₜ | Tₜ | F2 | Limit |
| No. | (Min) | (Reference) | (Test) | Value | 50-100 |
| 1 | 5 | 3 | 1 | 64 | F2 Value is |
| 2 | 10 | 19 | 16 | | within limit |
| 3 | 15 | 58 | 52 | | (Limit: 50-100) |
| 4 | 20 | 91 | 82 | | |
| 5 | 30 | 95 | 97 | | |
| 6 | 45 | 96 | 97 | | |
| 7 | 60 | 96 | 97 | | |

Conclusion: As per above result, the dissolution of the test product as per example, were found to be similar to reference product Pradaxa 150 mg.

We claim:

1. A pharmaceutical composition comprising dabigatran etexilate or salt thereof and glycerophosphoric acid, optionally along with one or more pharmaceutically acceptable excipient.

2. The composition as claimed in 1, wherein amount of dabigatran etexilate or salt thereof ranging from 10 mg to 300 mg.

3. The composition as claimed in claim 1, wherein amount of glycerophosphoric acid ranges from 5 to 40% by weight of the composition.

4. The composition as claimed in claim 1, wherein ratio of dabigatran etexilate or salt thereof to Glycerophosphoric acid ranges from 1:0.2 to 1:2.

5. The composition as claimed in claim 1, wherein composition is in the form of pellet, granule, tablet, capsule or bead.

6. The composition as claimed in claim 1, wherein one or more pharmaceutically acceptable excipient is selected from the group consisting of diluent, disintegrant, binder, antioxidant, channeling agent, anti-adherent, antifoaming agent, seal coating polymer, solvent or combinations thereof.

7. The one or more pharmaceutically acceptable excipient as claimed in claim 6, wherein diluent is Microcrystalline Cellulose, disintegrant is Crospovidone, binder is Acacia or Hydroxypropyl cellulose or combination thereof, antioxidant is butylated Hydroxytoluene or butylated Hydroxyanisole or combination thereof, channeling agent is Sucrose, anti-adherent is talc, antifoaming agent is Simethicone, seal coating polymer is hydroxyl propyl methyl cellulose, solvent is isopropyl alcohol or dichloromethane or water or combination thereof.

8. A process of manufacturing pharmaceutical composition comprising dabigatran etexilate or salt thereof comprising central core, optionally seal coting, sugar coating, drug layer and optionally seal coating wherein steps comprises
i. Mixing glycerophosphoric acid along with one or more pharmaceutically acceptable excipient selected from binder, disintegrant, diluent and solvent to form the central core.
ii. Optionally coating the central core in step (i) by using seal coating polymer to form the seal coat.
iii. Coating the central core in step (i) or seal coating in step (ii) by using pharmaceutically acceptable excipient selected from channelizing agent, anti-adherent and solvent to form the sugar coating.
iv. Coating or drug layering the sugar coat in step (iii) by using the dabigatran etexilate or salt thereof, antioxidant, antifoaming agent, binder and solvent to form the drug layering or coating.
v. Optionally coating the drug layering in step (iv) by using seal coating polymer to form the seal coat.

9. The process of manufacturing pharmaceutical composition as claimed in claim 8;
wherein central core is manufactured by either dry or wet granulation method.

10. The process of manufacturing pharmaceutical composition as claimed in claim 8;
wherein diluent is Microcrystalline Cellulose, disintegrant is Crospovidone, binder is Acacia or Hydroxypropyl cellulose or combination thereof, antioxidant is butylated Hydroxytoluene or butylated Hydroxyanisole or combination thereof, channeling agent is Sucrose, anti-adherent is talc, antifoaming agent is Simethicone, seal coating polymer is hydroxyl propyl methyl cellulose, solvent is isopropyl alcohol or dichloromethane or water or combination thereof.
